(19) United States
(12) Patent Application Publication
(10) Pub. No.: US 2011/0039301 A1
(43) Pub. Date: Feb. 17, 2011

(54) Polypeptides Having Organophosphorous Hydrolase Activity and Polynucleotides Encoding Same

(75) Inventors: Soeren Flensted Lassen, Farum (DK); Steffen Danielsen, CopenHagen Oe (DK); Lars Henrik Oestergaard, Charlottenlund (DK); Lars Kobberoee Skov, Ballerup (DK)

Correspondence Address: NOVOZYMES NORTH AMERICA, INC., 500 FIFTH AVENUE, SUITE 1600, NEW YORK, NY, 10110, US

(73) Assignee: Novozymes A/S, Bagsvaerd (DK)

(21) Appl. No.: 12/988,096

(22) PCT Filed: Apr. 23, 2009
(86) PCT No.: PCT/EP2009/054906

§ 371 Date: Oct. 15, 2010

Related U.S. Application Data
(60) Provisional application No. 61/047,798, filed on Apr. 25, 2008.

(30) Foreign Application Priority Data
Apr. 24, 2008 (EP) 08155075.8

(51) Int. Cl. C12N 9/14; C12N 15/55; C12N 15/63; C12N 1/00; C12P 21/00; A62D 3/02

(52) U.S. Cl. 435/69.1; 435/195; 536/23.2; 435/320.1; 435/243; 435/262.5

(57) ABSTRACT

The present invention relates to isolated polypeptides having organophosphorous hydrolase activity and isolated polynucleotides encoding the polypeptides. The invention also relates to nucleic acid constructs, vectors, and host cells comprising the polynucleotides as well as methods of producing and using the polypeptides.

### REFERENCE TO A SEQUENCE LISTING

This application contains a Sequence Listing in computer readable form. The computer readable form is incorporated herein by reference.

### BACKGROUND OF THE INVENTION

1. Field of the Invention

The present invention relates to isolated polypeptides having organophosphorous hydrolase activity and isolated polynucleotides encoding the polypeptides. The invention also relates to nucleic acid constructs, vectors, and host cells comprising the polynucleotides as well as methods of producing and using the polypeptides.

2. Description of the Related Art

Organophosphorous compounds are known in the art. In particular some warfare agents are known to be organophosphorous compounds such as Sarin, Cyclosarin, and Soman. Other organophosphorous compounds are known as pesticides.

It is desirable to be able to decontaminate areas contaminated with such organophosphorous compounds. A polypeptide having organophosphorous hydrolase activity has been suggested for this purpose since such polypeptides are capable of hydrolyzing such harmful organophosphorous compounds and thereby converting them to less harmful products.

Organophosphorous hydrolases are known in the art.

Cheng et al. (1997), (J. Ind Microbiol. Biotechnol. 18:49-55) disclose the nucleotide sequence of an organophosphorous hydrolase derived from Pseudoalteromonas haloplanktis (Alteromonas haloplanktis).

It is an object of the present invention to provide polypeptides having organophosphorous hydrolase activity and polynucleotides encoding the polypeptides, in particular having high stability and for high specific activity.

### SUMMARY OF THE INVENTION

The present invention relates to isolated polypeptides having organophosphorous hydrolase activity selected from the group consisting of:

(a) a polypeptide comprising an amino acid sequence having at least 92% identity to the mature polypeptide of SEQ ID NO: 2;
(b) a polypeptide encoded by a polynucleotide that hybridizes under at least high stringency conditions with (i) the mature polypeptide coding sequence of SEQ ID NO: 1, (ii) the genomic sequence encoding the mature polypeptide coding sequence of SEQ ID NO: 1, or (iii) a full-length complementary strand of (i) or (ii);
(c) a polypeptide encoded by a polynucleotide comprising a nucleotide sequence having at least 92% identity to the mature polypeptide coding sequence of SEQ ID NO: 1; and
(d) a variant comprising a substitution, deletion, and/or insertion of one or more (several) amino acids of the mature polypeptide of SEQ ID NO: 2.

The present invention also relates to isolated polynucleotides encoding polypeptides having organophosphorous hydrolase activity, selected from the group consisting of:

(a) a polynucleotide encoding a polypeptide comprising an amino acid sequence having at least 92% identity to the mature polypeptide of SEQ ID NO: 2;
(b) a polynucleotide that hybridizes under at least high stringency conditions with (i) the mature polypeptide coding sequence of SEQ ID NO: 1, (ii) the genomic DNA sequence encoding the mature polypeptide coding sequence of SEQ ID NO: 1, or (iii) a full-length complementary strand of (i) or (ii);
(c) a polynucleotide comprising a nucleotide sequence having at least 92% identity to the mature polypeptide coding sequence of SEQ ID NO: 1; and
(d) a polynucleotide encoding a variant comprising a substitution, deletion, and/or insertion of one or more (several) amino acids of the mature polypeptide of SEQ ID NO: 2.

The present invention also relates to nucleic acid constructs, recombinant expression vectors, recombinant host cells comprising the polynucleotides, and methods of producing a polypeptide having organophosphorous hydrolase activity.

The present invention also relates to methods of inhibiting the expression of a polypeptide in a cell, comprising administering to the cell or expressing in the cell a double-stranded RNA (dsRNA) molecule, wherein the dsRNA comprises a subsequence of a polynucleotide of the present invention. The present also relates to such a double-stranded inhibitory RNA (dsRNA) molecule, wherein optionally the dsRNA is an siRNA or an miRNA molecule.

The present invention also relates to methods of degrading organophosphorous compounds.

In particular the present invention relation to methods of decontaminating an area or a device contaminated with one or more hazardous or undesired organophosphorous compounds by applying the organophosphorous hydrolase of the invention to said area or device.

The present invention also relates to plants comprising an isolated polynucleotide encoding such a polypeptide having organophosphorous hydrolase activity.

The present invention also relates to methods of producing such a polypeptide having organophosphorous hydrolase activity, comprising: (a) cultivating a transgenic plant or a plant cell comprising a polynucleotide encoding such a polypeptide having organophosphorous hydrolase activity under conditions conducive for production of the polypeptide; and (b) recovering the polypeptide.

### BRIEF DESCRIPTION OF THE FIGURES

FIG. 1 shows the pH profile of the organophosphorous hydrolase of the invention as described in Example 4.

FIG. 2 discloses stability data for the organophosphorous hydrolase of the invention.

### DEFINITIONS

Organophosphorous hydrolase activity: The term “organophosphorous hydrolase activity” is defined herein as a hydrolytic activity (EC 3.1.8.1) that catalyzes the hydrolysis of organophosphorus compounds. For purposes of the present invention, organiphosphorous hydrolase activity is determined according to the procedure described in the commercial kit EnzChek® Paraoxonase Assay Kit from Invitrogen. EnzChek Paraoxonase Assay Kit manual (version 04-24-2006) http://probes.invitrogen.com/media/pis/mp33702.pdf. One unit of organophosphorous hydrolase activity equals the amount of enzyme capable of releasing 1 μmole of organophosphate per minute at pH 8.0, 25° C.

The polypeptides of the present invention have at least 20%, preferably at least 40%, more preferably at least 50%, more preferably at least 60%, more preferably at least 70%, more preferably at least 80%, even more preferably at least 90%, most preferably at least 95%, and even most preferably at least 100% of the organophosphorous hydrolase activity of the mature polypeptide of SEQ ID NO: 2.

Isolated polypeptide: The term “isolated polypeptide” as used herein refers to a polypeptide that is isolated from a source. In a preferred aspect, the polypeptide is at least 1% pure, preferably at least 5% pure, more preferably at least 10% pure, more preferably at least 20% pure, more preferably at least 40% pure, more preferably at least 60% pure, even more preferably at least 80% pure, and most preferably at least 90% pure, as determined by SDS-PAGE.

Substantially pure polypeptide: The term “substantially pure polypeptide” denotes herein a polypeptide preparation that contains at most 10%, preferably at most 8%, more preferably at most 6%, more preferably at most 5%, more preferably at most 4%, more preferably at most 3%, even more preferably at most 2%, most preferably at most 1%, and even most preferably at most 0.5% by weight of other polypeptide material with which it is natively or recombinantly associated. It is, therefore, preferred that the substantially pure polypeptide is at least 92% pure, preferably at least 94% pure, more preferably at least 95% pure, more preferably at least 96% pure, more preferably at least 96% pure, more preferably at least 97% pure, more preferably at least 98% pure, even more preferably at least 99%, most preferably at least 99.5% pure, and even most preferably 100% pure by weight of the total polypeptide material present in the preparation. The polypeptides of the present invention are preferably in a substantially pure form, i.e., that the polypeptide preparation is essentially free of other polypeptide material with which it is natively or recombinantly associated. This can be accomplished, for example, by preparing the polypeptide by well-known recombinant methods or by classical purification methods.

Mature polypeptide: The term “mature polypeptide” is defined herein as a polypeptide having organophosphorous hydrolase activity that is in its final form following translation and any post-translational modifications, such as N-terminal processing, C-terminal truncation, glycosylation, phosphorylation, etc.

Mature polypeptide coding sequence: The term “mature polypeptide coding sequence” is defined herein as a nucleotide sequence that encodes a mature polypeptide having organophosphorous hydrolase activity.

Identity: The relatedness between two amino acid sequences or between two nucleotide sequences is described by the parameter “identity”.

For purposes of the present invention, the degree of identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends in Genetics 16: 276-277), preferably version 3.0.0 or later. The optional parameters used are gap open penalty of 10, gap extension penalty of 0.5, and the EBLOSUM62 (EMBOSS version of BLOSUM62) substitution matrix. The output of Needle labeled “longest identity” (obtained using the—nobrief option) is used as the percent identity and is calculated as follows:

(Identical Residues×100)/(Length of Alignment−Total Number of Gaps in Alignment)

For purposes of the present invention, the degree of identity between two deoxyribonucleotide sequences is determined using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), preferably version 3.0.0 or later. The optional parameters used are gap open penalty of 10, gap extension penalty of 0.5, and the EDNAFULL (EMBOSS version of NCBI NUC4.4) substitution matrix. The output of Needle labeled “longest identity” (obtained using the—nobrief option) is used as the percent identity and is calculated as follows:

(Identical Deoxyribonucleotides×100)/(Length of Alignment−Total Number of Gaps in Alignment)

Homologous sequence: The term “homologous sequence” is defined herein as a predicted protein that gives an E value (or expectancy score) of less than 0.001 in a tfasty search (Pearson, W. R., 1999, in Bioinformatics Methods and Protocols, S. Misener and S. A. Krawetz, ed., pp. 185-219) with the Pseudoalteromonas haloplanktis NCIMB 1964 organophosphorous hydrolase.

Polypeptide fragment: The term “polypeptide fragment” is defined herein as a polypeptide having one or more (several) amino acids deleted from the amino and/or carboxyl terminus of the mature polypeptide of SEQ ID NO: 2; or a homologous sequence thereof; wherein the fragment has organophosphorous hydrolase activity.

Subsequence: The term “subsequence” is defined herein as a nucleotide sequence having one or more (several) nucleotides deleted from the 5′ and/or 3′ end of the mature polypeptide coding sequence of SEQ ID NO: 1; or a homologous sequence thereof; wherein the subsequence encodes a polypeptide fragment having organophosphorous hydrolase activity

Allelic variant: The term “allelic variant” denotes herein any of two or more alternative forms of a gene occupying the same chromosomal locus. Allelic variation arises naturally through mutation, and may result in polymorphism within populations. Gene mutations can be silent (no change in the encoded polypeptide) or may encode polypeptides having altered amino acid sequences. An allelic variant of a polypeptide is a polypeptide encoded by an allelic variant of a gene.

Isolated polynucleotide: The term “isolated polynucleotide” as used herein refers to a polynucleotide that is isolated from a source. In a preferred aspect, the polynucleotide is at least 1% pure, preferably at least 5% pure, more preferably at least 10% pure, more preferably at least 20% pure, more preferably at least 40% pure, more preferably at least 60% pure, even more preferably at least 80% pure, and most preferably at least 90% pure, as determined by agarose electrophoresis.

Substantially pure polynucleotide: The term “substantially pure polynucleotide” as used herein refers to a polynucleotide preparation free of other extraneous or unwanted nucleotides and in a form suitable for use within genetically engineered protein production systems. Thus, a substantially pure polynucleotide contains at most 10%, preferably at most 8%, more preferably at most 6%, more preferably at most 5%, more preferably at most 4%, more preferably at most 3%, even more preferably at most 2%, most preferably at most 1%, and even most preferably at most 0.5% by weight of other polynucleotide material with which it is natively or recombinantly associated. A substantially pure polynucleotide may, however, include naturally occurring 5′ and 3′ untranslated regions, such as promoters and terminators. It is preferred that the substantially pure polynucleotide is at least 90% pure, preferably at least 92% pure, more preferably at least 94% pure, more preferably at least 95% pure, more preferably at least 96% pure, more preferably at least 97% pure, even more preferably at least 98% pure, most preferably at least 99%, and even most preferably at least 99.5% pure by weight. The polynucleotides of the present invention are preferably in a substantially pure form, i.e., that the polynucleotide preparation is essentially free of other polynucleotide material with which it is natively or recombinantly associated. The polynucleotides may be of genomic, cDNA, RNA, semisynthetic, synthetic origin, or any combinations thereof.

Coding sequence: When used herein the term “coding sequence” means a nucleotide sequence, which directly specifies the amino acid sequence of its protein product. The boundaries of the coding sequence are generally determined by an open reading frame, which usually begins with the ATG start codon or alternative start codons such as GTG and TTG and ends with a stop codon such as TAA, TAG, and TGA. The coding sequence may be a DNA, cDNA, synthetic, or recombinant nucleotide sequence.

Nucleic acid construct: The term “nucleic acid construct” as used herein refers to a nucleic acid molecule, either single- or double-stranded, which is isolated from a naturally occurring gene or which is modified to contain segments of nucleic acids in a manner that would not otherwise exist in nature or which is synthetic. The term nucleic acid construct is synonymous with the term “expression cassette” when the nucleic acid construct contains the control sequences required for expression of a coding sequence of the present invention.

Control sequences: The term “control sequences” is defined herein to include all components necessary for the expression of a polynucleotide encoding a polypeptide of the present invention. Each control sequence may be native or foreign to the nucleotide sequence encoding the polypeptide or native or foreign to each other. Such control sequences include, but are not limited to, a leader, polyadenylation sequence, propeptide sequence, promoter, signal peptide sequence, and transcription terminator. At a minimum, the control sequences include a promoter, and transcriptional and translational stop signals. The control sequences may be provided with linkers for the purpose of introducing specific restriction sites facilitating ligation of the control sequences with the coding region of the nucleotide sequence encoding a polypeptide.

Operably linked: The term “operably linked” denotes herein a configuration in which a control sequence is placed at an appropriate position relative to the coding sequence of the polynucleotide sequence such that the control sequence directs the expression of the coding sequence of a polypeptide.

Expression: The term “expression” includes any step involved in the production of the polypeptide including, but not limited to, transcription, post-transcriptional modification, translation, post-translational modification, and secretion.

Expression vector: The term “expression vector” is defined herein as a linear or circular DNA molecule that comprises a polynucleotide encoding a polypeptide of the present invention and is operably linked to additional nucleotides that provide for its expression.

Host cell: The term “host cell”, as used herein, includes any cell type that is susceptible to transformation, transfection, transduction, and the like with a nucleic acid construct or expression vector comprising a polynucleotide of the present invention.

Modification: The term “modification” means herein any chemical modification of the polypeptide consisting of the mature polypeptide of SEQ ID NO: 2; or a homologous sequence thereof; as well as genetic manipulation of the DNA encoding such a polypeptide. The modification can be a substitution, a deletion and/or an insertion of one or more (several) amino acids as well as replacements of one or more (several) amino acid side chains.

Artificial variant: When used herein, the term “artificial variant” means a polypeptide having organophosphorous hydrolase activity produced by an organism expressing a modified polynucleotide sequence of the mature polypeptide coding sequence of SEQ ID NO: 1; or a homologous sequence thereof. The modified nucleotide sequence is obtained through human intervention by modification of the polynucleotide sequence disclosed in SEQ ID NO: 1; or a homologous sequence thereof.

### DETAILED DESCRIPTION OF THE INVENTION

### Polypeptides Having Organophosphorous Hydrolase Activity

In a first aspect, the present invention relates to isolated polypeptides comprising an amino acid sequence having a degree of identity to the mature polypeptide of SEQ ID NO: 2 of preferably at least 92%, more preferably at least 93%, more preferably at least 94%, more preferably at least 95%, more preferably at least 96%, more preferably at least 97%, more preferably at least 98%, and even most preferably at least 99%, which have organophosphorous hydrolase activity (hereinafter “homologous polypeptides”). In a preferred aspect, the homologous polypeptides have an amino acid sequence that differs by ten amino acids, preferably by five amino acids, more preferably by four amino acids, even more preferably by three amino acids, most preferably by two amino acids, and even most preferably by one amino acid from the mature polypeptide of SEQ ID NO: 2.

A polypeptide of the present invention preferably comprises the amino acid sequence of SEQ ID NO: 2 or an allelic variant thereof; or a fragment thereof having organophosphorous hydrolase activity. In a preferred aspect, the polypeptide comprises the amino acid sequence of SEQ ID NO: 2. In another preferred aspect, the polypeptide comprises the mature polypeptide of SEQ ID NO: 2.

In another preferred aspect, the polypeptide consists of the amino acid sequence of SEQ ID NO: 2 or an allelic variant thereof; or a fragment thereof having organophosphorous hydrolase activity. In another preferred aspect, the polypeptide consists of the amino acid sequence of SEQ ID NO: 2.

In a second aspect, the present invention relates to isolated polypeptides having organophosphorous hydrolase activity that are encoded by polynucleotides that hybridize under preferably very low stringency conditions, more preferably low stringency conditions, more preferably medium stringency conditions, more preferably medium-high stringency conditions, even more preferably high stringency conditions, and most preferably very high stringency conditions with (i) the mature polypeptide coding sequence of SEQ ID NO: 1, (ii) the genomic DNA comprising the mature polypeptide coding sequence of SEQ ID NO: 1, (iii) a subsequence of (i) or (ii), or (iv) a full-length complementary strand of (i), (ii), or (iii) (J. Sambrook, E. F. Fritsch, and T. Maniatis, 1989, Molecular Cloning, A Laboratory Manual, 2d edition, Cold Spring Harbor, N.Y.). A subsequence of the mature polypeptide coding sequence of SEQ ID NO: 1 contains at least 100 contiguous nucleotides or preferably at least 200 contiguous nucleotides. Moreover, the subsequence may encode a polypeptide fragment having organophosphorous hydrolase activity. In a preferred aspect, the complementary strand is the full-length complementary strand of the mature polypeptide coding sequence of SEQ ID NO: 1.

The nucleotide sequence of SEQ ID NO: 1; or a subsequence thereof; as well as the amino acid sequence of SEQ ID NO: 2; or a fragment thereof; may be used to design nucleic acid probes to identify and clone DNA encoding polypeptides having organophosphorous hydrolase activity from strains of different genera or species according to methods well known in the art. In particular, such probes can be used for hybridization with the genomic or cDNA of the genus or species of interest, following standard Southern blotting procedures, in order to identify and isolate the corresponding gene therein. Such probes can be considerably shorter than the entire sequence, but should be at least 14, preferably at least 25, more preferably at least 35, and most preferably at least 70 nucleotides in length. It is, however, preferred that the nucleic acid probe is at least 100 nucleotides in length. For example, the nucleic acid probe may be at least 200 nucleotides, preferably at least 300 nucleotides, more preferably at least 400 nucleotides, or most preferably at least 500 nucleotides in length. Even longer probes may be used, e.g., nucleic acid probes that are preferably at least 600 nucleotides, more preferably at least 700 nucleotides, even more preferably at least 800 nucleotides, or most preferably at least 900 nucleotides in length. Both DNA and RNA probes can be used. The probes are typically labeled for detecting the corresponding gene (for example, with ³²P, ³H, ³⁵S, biotin, or avidin). Such probes are encompassed by the present invention.

A genomic DNA library prepared from such other strains may, therefore, be screened for DNA that hybridizes with the probes described above and encodes a polypeptide having organophosphorous hydrolase activity. Genomic or other DNA from such other strains may be separated by agarose or polyacrylamide gel electrophoresis, or other separation techniques. DNA from the libraries or the separated DNA may be transferred to and immobilized on nitrocellulose or other suitable carrier material. In order to identify a clone or DNA that is homologous with SEQ ID NO: 1; or a subsequence thereof; the carrier material is preferably used in a Southern blot.

For purposes of the present invention, hybridization indicates that the nucleotide sequence hybridizes to a labeled nucleic acid probe corresponding to the mature polypeptide coding sequence of SEQ ID NO: 1; the genomic DNA comprising the mature polypeptide coding sequence of SEQ ID NO: 1; its full-length complementary strand; or a subsequence thereof; under very low to very high stringency conditions. Molecules to which the nucleic acid probe hybridizes under these conditions can be detected using, for example, X-ray film.

In a preferred aspect, the nucleic acid probe is the mature polypeptide coding sequence of SEQ ID NO: 1. In another preferred aspect, the nucleic acid probe is a polynucleotide sequence that encodes the polypeptide of SEQ ID NO: 2, or a subsequence thereof. In another preferred aspect, the nucleic acid probe is SEQ ID NO: 1. In another preferred aspect, the nucleic acid probe is the polynucleotide sequence contained in plasmid pOPAANN10719-1, wherein the polynucleotide sequence thereof encodes a polypeptide having organophosphorous hydrolase activity. In another preferred aspect, the nucleic acid probe is the mature polypeptide coding region contained in plasmid pOPAANN10719-1. For long probes of at least 100 nucleotides in length, very low to very high stringency conditions are defined as prehybridization and hybridization at 42° C. in 5×SSPE, 0.3% SDS, 200 μg/mL sheared and denatured salmon sperm DNA, and 50% formamide for high and very high stringencies, following standard Southern blotting procedures for 12 to 24 hours optimally. For long probes of at least 100 nucleotides in length, the carrier material is finally washed three times each for 15 minutes using 2×SSC, 0.2% SDS preferably at 65° C. (high stringency), and most preferably at 70° C. (very high stringency).

For short probes that are about 15 nucleotides to about 70 nucleotides in length, stringency conditions are defined as prehybridization, hybridization, and washing post-hybridization at about 5° C. to about 10° C. below the calculated Tₘ using the calculation according to Bolton and McCarthy (1962, Proceedings of the National Academy of Sciences USA 48:1390) in 0.9 M NaCl, 0.09 M Tris-HCl pH 7.6, 6 mM EDTA, 0.5% NP-40, 1×Denhardt's solution, 1 mM sodium pyrophosphate, 1 mM sodium monobasic phosphate, 0.1 mM ATP, and 0.2 mg of yeast RNA per mL following standard Southern blotting procedures for 12 to 24 hours optimally.

For short probes that are about 15 nucleotides to about 70 nucleotides in length, the carrier material is washed once in 6×SCC plus 0.1% SDS for 15 minutes and twice each for 15 minutes using 6×SSC at 5° C. to 10° C. below the calculated Tₘ.

In a third aspect, the present invention relates to isolated polypeptides having organophosphorous hydrolase activity encoded by polynucleotides comprising or consisting of nucleotide sequences that have a degree of identity to the mature polypeptide coding sequence of SEQ ID NO: 1 of preferably at least 90%, more preferably at least 91%, more preferably at least 92%, more preferably at least 93%, more preferably at least 94%, more preferably at least 95%, even more preferably at least 96%, most preferably at least 97%, and even most preferably 98%, or 99%, which encode an active polypeptide. See polynucleotide section herein.

In a fourth aspect, the present invention relates to artificial variants comprising a substitution, deletion, and/or insertion of one or more (or several) amino acids of the mature polypeptide of SEQ ID NO: 2; or a homologous sequence thereof. Preferably, amino acid changes are of a minor nature, that is conservative amino acid substitutions or insertions that do not significantly affect the folding and/or activity of the protein; small deletions, typically of one to about 30 amino acids; small amino- or carboxyl-terminal extensions, such as an amino-terminal methionine residue; a small linker peptide of up to about 20-25 residues; or a small extension that facilitates purification by changing net charge or another function, such as a poly-histidine tract, an antigenic epitope or a binding domain.

Examples of conservative substitutions are within the group of basic amino acids (arginine, lysine and histidine), acidic amino acids (glutamic acid and aspartic acid), polar amino acids (glutamine and asparagine), hydrophobic amino acids (leucine, isoleucine and valine), aromatic amino acids (phenylalanine, tryptophan and tyrosine), and small amino acids (glycine, alanine, serine, threonine and methionine). Amino acid substitutions that do not generally alter specific activity are known in the art and are described, for example, by H. Neurath and R. L. Hill, 1979, In, The Proteins, Academic Press, New York. The most commonly occurring exchanges are Ala/Ser, Val/Ile, Asp/Glu, Thr/Ser, Ala/Gly, Ala/Thr, Ser/Asn, Ala/Val, Ser/Gly, Tyr/Phe, Ala/Pro, Lys/Arg, Asp/Asn, Leu/Ile, Leu/Val, Ala/Glu, and Asp/Gly.

In addition to the 20 standard amino acids, non-standard amino acids (such as 4-hydroxyproline, 6-N-methyl lysine, 2-aminoisobutyric acid, isovaline, and alpha-methyl serine) may be substituted for amino acid residues of a wild-type polypeptide. A limited number of non-conservative amino acids, amino acids that are not encoded by the genetic code, and unnatural amino acids may be substituted for amino acid residues. “Unnatural amino acids” have been modified after protein synthesis, and/or have a chemical structure in their side chain(s) different from that of the standard amino acids. Unnatural amino acids can be chemically synthesized, and preferably, are commercially available, and include pipecolic acid, thiazolidine carboxylic acid, dehydroproline, 3- and 4-methylproline, and 3,3-dimethylproline.

Alternatively, the amino acid changes are of such a nature that the physico-chemical properties of the polypeptides are altered. For example, amino acid changes may improve the thermal stability of the polypeptide, alter the substrate specificity, change the pH optimum, and the like.

Essential amino acids in the parent polypeptide can be identified according to procedures known in the art, such as site-directed mutagenesis or alanine-scanning mutagenesis (Cunningham and Wells, 1989, Science 244: 1081-1085). In the latter technique, single alanine mutations are introduced at every residue in the molecule, and the resultant mutant molecules are tested for biological activity (i.e., organophosphorous hydrolase activity) to identify amino acid residues that are critical to the activity of the molecule. See also, Hilton et al., 1996, J. Biol. Chem. 271: 4699-4708. The active site of the enzyme or other biological interaction can also be determined by physical analysis of structure, as determined by such techniques as nuclear magnetic resonance, crystallography, electron diffraction, or photoaffinity labeling, in conjunction with mutation of putative contact site amino acids. See, for example, de Vos et al., 1992, Science 255: 306-312; Smith et al., 1992, J. Mol. Biol. 224: 899-904; Wlodaver et al., 1992, FEBS Lett. 309: 59-64. The identities of essential amino acids can also be inferred from analysis of identities with polypeptides that are related to a polypeptide according to the invention.

Single or multiple amino acid substitutions, deletions, and/or insertions can be made and tested using known methods of mutagenesis, recombination, and/or shuffling, followed by a relevant screening procedure, such as those disclosed by Reidhaar-Olson and Sauer, 1988, Science 241: 53-57; Bowie and Sauer, 1989, Proc. Natl. Acad. Sci. USA 86: 2152-2156; WO 95/17413; or WO 95/22625. Other methods that can be used include error-prone PCR, phage display (e.g., Lowman et al., 1991, Biochem. 30: 10832-10837; U.S. Pat. No. 5,223,409; WO 92/06204), and region-directed mutagenesis (Derbyshire et al., 1986, Gene 46: 145; Ner et al., 1988, DNA 7: 127).

Mutagenesis/shuffling methods can be combined with high-throughput, automated screening methods to detect activity of cloned, mutagenized polypeptides expressed by host cells (Ness et al., 1999, Nature Biotechnology 17: 893-896). Mutagenized DNA molecules that encode active polypeptides can be recovered from the host cells and rapidly sequenced using standard methods in the art. These methods allow the rapid determination of the importance of individual amino acid residues in a polypeptide of interest, and can be applied to polypeptides of unknown structure.

The total number of amino acid substitutions, deletions and/or insertions of the mature polypeptide of SEQ ID NO: 2, is 10, preferably 9, more preferably 8, more preferably 7, more preferably at most 6, more preferably 5, more preferably 4, even more preferably 3, most preferably 2, and even most preferably 1.

### Sources of Polypeptides Having Organophosphorous Hydrolase Activity

A polypeptide of the present invention may be obtained from microorganisms of any genus. For purposes of the present invention, the term “obtained from” as used herein in connection with a given source shall mean that the polypeptide encoded by a nucleotide sequence is produced by the source or by a strain in which the nucleotide sequence from the source has been inserted. In a preferred aspect, the polypeptide obtained from a given source is secreted extracellularly.

A polypeptide having organophosphorous hydrolase activity of the present invention may be a bacterial polypeptide. For example, the polypeptide may be a gram positive bacterial polypeptide such as a Bacillus, Streptococcus, Streptomyces, Staphylococcus, Enterococcus, Lactobacillus, Lactococcus, Clostridium, Geobacillus, or Oceanobacillus polypeptide having organophosphorous hydrolase activity, or a Gram negative bacterial polypeptide such as an E. coli, Pseudomonas, Salmonella, Campylobacter, Helicobacter, Flavobacterium, Fusobacterium, Ilyobacter, Neisseria, or Ureaplasma polypeptide having organophosphorous hydrolase activity.

In a preferred aspect, the polypeptide is a Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus pumilus, Bacillus stearothermophilus, Bacillus subtilis, or Bacillus thuringiensis polypeptide having organophosphorous hydrolase activity.

In another preferred aspect, the polypeptide is a Streptococcus equisimilis, Streptococcus pyogenes, Streptococcus uberis, or Streptococcus equi subsp. Zooepidemicus polypeptide having organophosphorous hydrolase activity.

In another preferred aspect, the polypeptide is a Streptomyces achromogenes, Streptomyces avermitilis, Streptomyces coelicolor, Streptomyces griseus, or Streptomyces lividans polypeptide having organophosphorous hydrolase activity.

In another preferred aspect, the polypeptide is a Pseudoalteramonas haloplanktis polypeptide.

In a more preferred aspect, the polypeptide is a Pseudoalteromonas haloplanktis polypeptide having organophosphorous hydrolase activity. In a most preferred aspect, the polypeptide is a a Pseudoalteromonas haloplanktis NCIMB 1964 polypeptide having organophosphorous hydrolase activity, e.g., the polypeptide comprising the mature polypeptide of SEQ ID NO: 2.

It will be understood that for the aforementioned species the invention encompasses both the perfect and imperfect states, and other taxonomic equivalents, e.g., anamorphs, regardless of the species name by which they are known. Those skilled in the art will readily recognize the identity of appropriate equivalents.

Strains of these species are readily accessible to the public in a number of culture collections, such as the American Type Culture Collection (ATCC), Deutsche Sammlung von Mikroorganismen and Zellkulturen GmbH (DSM), Centraalbureau Voor Schimmelcultures (CBS), Agricultural Research Service Patent Culture Collection, Northern Regional Research Center (NRRL) and NCIMB.

Furthermore, such polypeptides may be identified and obtained from other sources including microorganisms isolated from nature (e.g., soil, composts, water, etc.) using the above-mentioned probes. Techniques for isolating microorganisms from natural habitats are well known in the art. The polynucleotide may then be obtained by similarly screening a genomic or cDNA library of such a microorganism. Once a polynucleotide sequence encoding a polypeptide has been detected with the probe(s), the polynucleotide can be isolated or cloned by utilizing techniques that are well known to those of ordinary skill in the art (see, e.g., Sambrook et al., 1989, supra).

Polypeptides of the present invention also include fused polypeptides or cleavable fusion polypeptides in which another polypeptide is fused at the N-terminus or the C-terminus of the polypeptide or fragment thereof. A fused polypeptide is produced by fusing a nucleotide sequence (or a portion thereof) encoding another polypeptide to a nucleotide sequence (or a portion thereof) of the present invention. Techniques for producing fusion polypeptides are known in the art, and include ligating the coding sequences encoding the polypeptides so that they are in frame and that expression of the fused polypeptide is under control of the same promoter(s) and terminator.

A fusion polypeptide can further comprise a cleavage site. Upon secretion of the fusion protein, the site is cleaved releasing the polypeptide having organophosphorous hydrolase activity from the fusion protein. Examples of cleavage sites include, but are not limited to, a Kex2 site that encodes the dipeptide Lys-Arg (Martin et al., 2003, J. Ind. Microbiol. Biotechnol. 3: 568-76; Svetina et al., 2000, J. Biotechnol. 76: 245-251; Rasmussen-Wilson et al., 1997, Appl. Environ. Microbiol. 63: 3488-3493; Ward et al., 1995, Biotechnology 13: 498-503; and Contreras et al., 1991, Biotechnology 9: 378-381), an Ile-(Glu or Asp)-Gly-Arg site, which is cleaved by a Factor Xa protease after the arginine residue (Eaton et al., 1986, Biochem. 25: 505-512); a Asp-Asp-Asp-Asp-Lys site, which is cleaved by an enterokinase after the lysine (Collins-Racie et al., 1995, Biotechnology 13: 982-987); a His-Tyr-Glu site or His-Tyr-Asp site, which is cleaved by Genenase I (Carter et al., 1989, Proteins: Structure, Function, and Genetics 6: 240-248); a Leu-Val-Pro-Arg-Gly-Ser site, which is cleaved by thrombin after the Arg (Stevens, 2003, Drug Discovery World 4: 35-48); a Glu-Asn-Leu-Tyr-Phe-Gln-Gly site, which is cleaved by TEV protease after the Gln (Stevens, 2003, supra); and a Leu-Glu-Val-Leu-Phe-Gln-Gly-Pro site, which is cleaved by a genetically engineered form of human rhinovirus 3C protease after the Gln (Stevens, 2003, supra).

### Polynucleotides

The present invention also relates to isolated polynucleotides comprising or consisting of nucleotide sequences that encode polypeptides having organophosphorous hydrolase activity of the present invention.

In a preferred aspect, the nucleotide sequence comprises or consists of SEQ ID NO: 1. In another preferred aspect, the nucleotide sequence comprises or consists of the mature polypeptide coding sequence of SEQ ID NO: 1. The present invention also encompasses nucleotide sequences that encode polypeptides comprising or consisting of the amino acid sequence of SEQ ID NO: 2 or the mature polypeptide thereof, which differ from SEQ ID NO: 1 or the mature polypeptide coding sequence thereof by virtue of the degeneracy of the genetic code. The present invention also relates to subsequences of SEQ ID NO: 1 that encode fragments of SEQ ID NO: 2 that have organophosphorous hydrolase activity.

The present invention also relates to mutant polynucleotides comprising or consisting of at least one mutation in the mature polypeptide coding sequence of SEQ ID NO: 1, in which the mutant nucleotide sequence encodes the mature polypeptide of SEQ ID NO: 2.

The techniques used to isolate or clone a polynucleotide encoding a polypeptide are known in the art and include isolation from genomic DNA, preparation from cDNA, or a combination thereof. The cloning of the polynucleotides of the present invention from such genomic DNA can be effected, e.g., by using the well known polymerase chain reaction (PCR) or antibody screening of expression libraries to detect cloned DNA fragments with shared structural features. See, e.g., Innis et al., 1990, PCR: A Guide to Methods and Application, Academic Press, New York. Other nucleic acid amplification procedures such as ligase chain reaction (LCR), ligated activated transcription (LAT) and nucleotide sequence-based amplification (NASBA) may be used. The polynucleotides may be cloned from a strain of Pseudoalteromonas, or another or related organism and thus, for example, may be an allelic or species variant of the polypeptide encoding region of the nucleotide sequence.

The present invention also relates to isolated polynucleotides comprising or consisting of nucleotide sequences that have a degree of identity to the mature polypeptide coding sequence of SEQ ID NO: 1 of 90%, more preferably at least 92%, more preferably at least 93%, more preferably at least 94%, more preferably at least 95% and even most preferably at least 96%, at least 97%, at least 98%, or at least 99% identity, which encode an active polypeptide.

Modification of a nucleotide sequence encoding a polypeptide of the present invention may be necessary for the synthesis of polypeptides substantially similar to the polypeptide. The term “substantially similar” to the polypeptide refers to non-naturally occurring forms of the polypeptide. These polypeptides may differ in some engineered way from the polypeptide isolated from its native source, e.g., artificial variants that differ in specific activity, thermostability, pH optimum, or the like. The variant sequence may be constructed on the basis of the nucleotide sequence presented as the mature polypeptide coding sequence of SEQ ID NO: 1, e.g., a subsequence thereof, and/or by introduction of nucleotide substitutions that do not give rise to another amino acid sequence of the polypeptide encoded by the nucleotide sequence, but which correspond to the codon usage of the host organism intended for production of the enzyme, or by introduction of nucleotide substitutions that may give rise to a different amino acid sequence. For a general description of nucleotide substitution, see, e.g., Ford et al., 1991, Protein Expression and Purification 2: 95-107.

It will be apparent to those skilled in the art that such substitutions can be made outside the regions critical to the function of the molecule and still result in an active polypeptide. Amino acid residues essential to the activity of the polypeptide encoded by an isolated polynucleotide of the invention, and therefore preferably not subject to substitution, may be identified according to procedures known in the art, such as site-directed mutagenesis or alanine-scanning mutagenesis (see, e.g., Cunningham and Wells, 1989, supra). In the latter technique, mutations are introduced at every positively charged residue in the molecule, and the resultant mutant molecules are tested for organophosphorous hydrolase activity to identify amino acid residues that are critical to the activity of the molecule. Sites of substrate-enzyme interaction can also be determined by analysis of the three-dimensional structure as determined by such techniques as nuclear magnetic resonance analysis, crystallography or photoaffinity labeling (see, e.g., de Vos et al., 1992, supra; Smith et al., 1992, supra; Wlodaver et al., 1992, supra).

The present invention also relates to isolated polynucleotides encoding polypeptides of the present invention, which hybridize under very low stringency conditions, preferably low stringency conditions, more preferably medium stringency conditions, more preferably medium-high stringency conditions, even more preferably high stringency conditions, and most preferably very high stringency conditions with (i) the mature polypeptide coding sequence of SEQ ID NO: 1, (ii) the genomic DNA sequence comprising the mature polypeptide coding sequence of SEQ ID NO: 1, or (iii) a full-length complementary strand of (i) or (ii); or allelic variants and subsequences thereof (Sambrook et al., 1989, supra), as defined herein. In a preferred aspect, the complementary strand is the full-length complementary strand of the mature polypeptide coding sequence of SEQ ID NO: 1.

The present invention also relates to isolated polynucleotides obtained by (a) hybridizing a population of DNA under very low, low, medium, medium-high, high, or very high stringency conditions with (i) the mature polypeptide coding sequence of SEQ ID NO: 1, (ii) the genomic DNA sequence comprising the mature polypeptide coding sequence of SEQ ID NO: 1, or (iii) a full-length complementary strand of (i) or (ii); and (b) isolating the hybridizing polynucleotide, which encodes a polypeptide having organophosphorous hydrolase activity. In a preferred aspect, the complementary strand is the full-length complementary strand of the mature polypeptide coding sequence of SEQ ID NO: 1.

### Nucleic Acid Constructs

The present invention also relates to nucleic acid constructs comprising an isolated polynucleotide of the present invention operably linked to one or more (several) control sequences that direct the expression of the coding sequence in a suitable host cell under conditions compatible with the control sequences.

An isolated polynucleotide encoding a polypeptide of the present invention may be manipulated in a variety of ways to provide for expression of the polypeptide. Manipulation of the polynucleotide's sequence prior to its insertion into a vector may be desirable or necessary depending on the expression vector. The techniques for modifying polynucleotide sequences utilizing recombinant DNA methods are well known in the art.

The control sequence may be an appropriate promoter sequence, a nucleotide sequence that is recognized by a host cell for expression of a polynucleotide encoding a polypeptide of the present invention. The promoter sequence contains transcriptional control sequences that mediate the expression of the polypeptide. The promoter may be any nucleotide sequence that shows transcriptional activity in the host cell of choice including mutant, truncated, and hybrid promoters, and may be obtained from genes encoding extracellular or intracellular polypeptides either homologous or heterologous to the host cell.

Examples of suitable promoters for directing the transcription of the nucleic acid constructs of the present invention, especially in a bacterial host cell, are the promoters obtained from the E. coli lac operon, Streptomyces coelicolor agarase gene (dagA), Bacillus subtilis levansucrase gene (sacB), Bacillus licheniformis alpha-amylase gene (amyL), Bacillus stearothermophilus maltogenic amylase gene (amyM), Bacillus amyloliquefaciens alpha-amylase gene (amyQ), Bacillus licheniformis penicillinase gene (penP), Bacillus subtilis xylA and xylB genes, and prokaryotic beta-lactamase gene (Villa-Kamaroff et al., 1978, Proceedings of the National Academy of Sciences USA 75: 3727-3731), as well as the tac promoter (DeBoer et al., 1983, Proceedings of the National Academy of Sciences USA 80: 21-25). Further promoters are described in “Useful proteins from recombinant bacteria” in Scientific American, 1980, 242: 74-94; and in Sambrook et al., 1989, supra.

The control sequence may also be a suitable transcription terminator sequence, a sequence recognized by a host cell to terminate transcription. The terminator sequence is operably linked to the 3′ terminus of the nucleotide sequence encoding the polypeptide. Any terminator that is functional in the host cell of choice may be used in the present invention.

The control sequence may also be a suitable leader sequence, a nontranslated region of an mRNA that is important for translation by the host cell. The leader sequence is operably linked to the 5′ terminus of the nucleotide sequence encoding the polypeptide. Any leader sequence that is functional in the host cell of choice may be used in the present invention.

The control sequence may also be a signal peptide coding sequence that codes for an amino acid sequence linked to the amino terminus of a polypeptide and directs the encoded polypeptide into the cell's secretory pathway. The 5′ end of the coding sequence of the nucleotide sequence may inherently contain a signal peptide coding sequence naturally linked in translation reading frame with the segment of the coding sequence that encodes the secreted polypeptide. Alternatively, the 5′ end of the coding sequence may contain a signal peptide coding sequence that is foreign to the coding sequence. The foreign signal peptide coding sequence may be required where the coding sequence does not naturally contain a signal peptide coding sequence. Alternatively, the foreign signal peptide coding sequence may simply replace the natural signal peptide coding sequence in order to enhance secretion of the polypeptide. However, any signal peptide coding sequence that directs the expressed polypeptide into the secretory pathway of a host cell of choice, i.e., secreted into a culture medium, may be used in the present invention.

Effective signal peptide coding sequences for bacterial host cells are the signal peptide coding sequences obtained from the genes for Bacillus NCIB 11837 maltogenic amylase, Bacillus stearothermophilus alpha-amylase, Bacillus licheniformis subtilisin, Bacillus licheniformis beta-lactamase, Bacillus stearothermophilus neutral proteases (nprT, nprS, nprM), Bacillus clausii alcaline protease (aprH) and Bacillus subtilis prsA. Further signal peptides are described by Simonen and Palva, 1993, Microbiological Reviews 57: 109-137.

The control sequence may also be a propeptide coding sequence that codes for an amino acid sequence positioned at the amino terminus of a polypeptide. The resultant polypeptide is known as a proenzyme or propolypeptide (or a zymogen in some cases). A propeptide is generally inactive and can be converted to a mature active polypeptide by catalytic or autocatalytic cleavage of the propeptide from the propolypeptide. The propeptide coding sequence may be obtained from the genes for Bacillus subtilis alkaline protease (aprE), Bacillus subtilis neutral protease (nprT), Saccharomyces cerevisiae alpha-factor, Rhizomucor miehei aspartic proteinase, and Myceliophthora thermophila laccase (WO 95/33836).

Where both signal peptide and propeptide sequences are present at the amino terminus of a polypeptide, the propeptide sequence is positioned next to the amino terminus of a polypeptide and the signal peptide sequence is positioned next to the amino terminus of the propeptide sequence.

It may also be desirable to add regulatory sequences that allow the regulation of the expression of the polypeptide relative to the growth of the host cell. Examples of regulatory systems are those that cause the expression of the gene to be turned on or off in response to a chemical or physical stimulus, including the presence of a regulatory compound. Regulatory systems in prokaryotic systems include the lac, tac, xyl and trp operator systems. Other examples of regulatory sequences are those that allow for gene amplification.

### Expression Vectors

The present invention also relates to recombinant expression vectors comprising a polynucleotide of the present invention, a promoter, and transcriptional and translational stop signals. The various nucleic acids and control sequences described herein may be joined together to produce a recombinant expression vector that may include one or more (several) convenient restriction sites to allow for insertion or substitution of the nucleotide sequence encoding the polypeptide at such sites. Alternatively, a polynucleotide sequence of the present invention may be expressed by inserting the nucleotide sequence or a nucleic acid construct comprising the sequence into an appropriate vector for expression. In creating the expression vector, the coding sequence is located in the vector so that the coding sequence is operably linked with the appropriate control sequences for expression.

The recombinant expression vector may be any vector (e.g., a plasmid or virus) that can be conveniently subjected to recombinant DNA procedures and can bring about expression of the nucleotide sequence. The choice of the vector will typically depend on the compatibility of the vector with the host cell into which the vector is to be introduced. The vectors may be linear or closed circular plasmids.

The vector may be an autonomously replicating vector, i.e., a vector that exists as an extrachromosomal entity, the replication of which is independent of chromosomal replication, e.g., a plasmid, an extrachromosomal element, a minichromosome, or an artificial chromosome. The vector may contain any means for assuring self-replication. Alternatively, the vector may be one that, when introduced into the host cell, is integrated into the genome and replicated together with the chromosome(s) into which it has been integrated. Furthermore, a single vector or plasmid or two or more vectors or plasmids that together contain the total DNA to be introduced into the genome of the host cell, or a transposon, may be used.

The vectors of the present invention preferably contain one or more (several) selectable markers that permit easy selection of transformed, transfected, transduced, or the like cells. A selectable marker is a gene the product of which provides for biocide or viral resistance, resistance to heavy metals, prototrophy to auxotrophs, and the like.

Examples of bacterial selectable markers are the dal genes from Bacillus subtilis or Bacillus licheniformis, or markers that confer antibiotic resistance such as ampicillin, kanamycin, chloramphenicol, or tetracycline resistance.

The vectors of the present invention preferably contain an element(s) that permits integration of the vector into the host cell's genome or autonomous replication of the vector in the cell independent of the genome.

For integration into the host cell genome, the vector may rely on the polynucleotide's sequence encoding the polypeptide or any other element of the vector for integration into the genome by homologous or nonhomologous recombination. Alternatively, the vector may contain additional nucleotide sequences for directing integration by homologous recombination into the genome of the host cell at a precise location(s) in the chromosome(s). To increase the likelihood of integration at a precise location, the integrational elements should preferably contain a sufficient number of nucleic acids, such as 100 to 10,000 base pairs, preferably 400 to 10,000 base pairs, and most preferably 800 to 10,000 base pairs, which have a high degree of identity to the corresponding target sequence to enhance the probability of homologous recombination. The integrational elements may be any sequence that is homologous with the target sequence in the genome of the host cell. Furthermore, the integrational elements may be non-encoding or encoding nucleotide sequences. On the other hand, the vector may be integrated into the genome of the host cell by non-homologous recombination.

For autonomous replication, the vector may further comprise an origin of replication enabling the vector to replicate autonomously in the host cell in question. The origin of replication may be any plasmid replicator mediating autonomous replication that functions in a cell. The term “origin of replication” or “plasmid replicator” is defined herein as a nucleotide sequence that enables a plasmid or vector to replicate in vivo.

Examples of bacterial origins of replication are the origins of replication of plasmids pBR322, pUC19, pACYC177, and pACYC184 permitting replication in E. coli, and pUB110, pE194, pTA1060, and pAMR1 permitting replication in Bacillus.

More than one copy of a polynucleotide of the present invention may be inserted into a host cell to increase production of the gene product. An increase in the copy number of the polynucleotide can be obtained by integrating at least one additional copy of the sequence into the host cell genome or by including an amplifiable selectable marker gene with the polynucleotide where cells containing amplified copies of the selectable marker gene, and thereby additional copies of the polynucleotide, can be selected for by cultivating the cells in the presence of the appropriate selectable agent.

The procedures used to ligate the elements described above to construct the recombinant expression vectors of the present invention are well known to one skilled in the art (see, e.g., Sambrook et al., 1989, supra).

### Host Cells

The present invention also relates to recombinant host cells, comprising an isolated polynucleotide of the present invention, which are advantageously used in the recombinant production of the polypeptides. A vector comprising a polynucleotide of the present invention is introduced into a host cell so that the vector is maintained as a chromosomal integrant or as a self-replicating extra-chromosomal vector as described earlier. The term “host cell” encompasses any progeny of a parent cell that is not identical to the parent cell due to mutations that occur during replication. The choice of a host cell will to a large extent depend upon the gene encoding the polypeptide and its source.

The host cell may be any cell useful in the recombinant production of a polypeptide of the present invention, e.g., a prokaryote or a eukaryote.

The prokaryotic host cell may be any Gram positive bacterium or a Gram negative bacterium. Gram positive bacteria include, but not limited to, Bacillus, Streptococcus, Streptomyces, Staphylococcus, Enterococcus, Lactobacillus, Lactococcus, Clostridium, Geobacillus, and Oceanobacillus. Gram negative bacteria include, but not limited to, E. coli, Pseudomonas, Salmonella, Campylobacter, Helicobacter, Flavobacterium, Fusobacterium, Ilyobacter, Neisseria, and Ureaplasma.

The bacterial host cell may be any Bacillus cell. Bacillus cells useful in the practice of the present invention include, but are not limited to, Bacillus alkalophilus, Bacillus amyloliquefaciens, Bacillus brevis, Bacillus circulans, Bacillus clausfi, Bacillus coagulans, Bacillus firmus, Bacillus lautus, Bacillus lentus, Bacillus licheniformis, Bacillus megaterium, Bacillus pumilus, Bacillus stearothermophilus, Bacillus subtilis, and Bacillus thuringiensis cells.

In a preferred aspect, the bacterial host cell is a Bacillus amyloliquefaciens, Bacillus lentus, Bacillus licheniformis, Bacillus stearothermophilus or Bacillus subtilis cell. In a more preferred aspect, the bacterial host cell is a Bacillus amyloliquefaciens cell. In another more preferred aspect, the bacterial host cell is a Bacillus clausii cell. In another more preferred aspect, the bacterial host cell is a Bacillus licheniformis cell. In another more preferred aspect, the bacterial host cell is a Bacillus subtilis cell.

The bacterial host cell may also be any Streptococcus cell. Streptococcus cells useful in the practice of the present invention include, but are not limited to, Streptococcus equisimilis, Streptococcus pyogenes, Streptococcus uberis, and Streptococcus equi subsp. Zooepidemicus cells.

In a preferred aspect, the bacterial host cell is a Streptococcus equisimilis cell. In another preferred aspect, the bacterial host cell is a Streptococcus pyogenes cell. In another preferred aspect, the bacterial host cell is a Streptococcus uberis cell. In another preferred aspect, the bacterial host cell is a Streptococcus equi subsp. Zooepidemicus cell.

The bacterial host cell may also be any Streptomyces cell. Streptomyces cells useful in the practice of the present invention include, but are not limited to, Streptomyces achromogenes, Streptomyces avermitilis, Streptomyces coelicolor, Streptomyces griseus, and Streptomyces lividans cells.

In a preferred aspect, the bacterial host cell is a Streptomyces achromogenes cell. In another preferred aspect, the bacterial host cell is a Streptomyces avermitilis cell. In another preferred aspect, the bacterial host cell is a Streptomyces coelicolor cell. In another preferred aspect, the bacterial host cell is a Streptomyces griseus cell. In another preferred aspect, the bacterial host cell is a Streptomyces lividans cell.

The introduction of DNA into a Bacillus cell may, for instance, be effected by protoplast transformation (see, e.g., Chang and Cohen, 1979, Molecular General Genetics 168: 111-115), by using competent cells (see, e.g., Young and Spizizen, 1961, Journal of Bacteriology 81: 823-829, or Dubnau and Davidoff-Abelson, 1971, Journal of Molecular Biology 56: 209-221), by electroporation (see, e.g., Shigekawa and Dower, 1988, Biotechniques 6: 742-751), or by conjugation (see, e.g., Koehler and Thorne, 1987, Journal of Bacteriology 169: 5271-5278). The introduction of DNA into an E. coli cell may, for instance, be effected by protoplast transformation (see, e.g., Hanahan, 1983, J. Mol. Biol. 166: 557-580) or electroporation (see, e.g., Dower et al., 1988, Nucleic Acids Res. 16: 6127-6145). The introduction of DNA into a Streptomyces cell may, for instance, be effected by protoplast transformation and electroporation (see, e.g., Gong et al., 2004, Folia Microbiol. (Praha) 49: 399-405), by conjugation (see, e.g., Mazodier et al., 1989, J. Bacteriol. 171: 3583-3585), or by transduction (see, e.g., Burke et al., 2001, Proc. Natl. Acad. Sci. USA 98: 6289-6294). The introduction of DNA into a Pseudomonas cell may, for instance, be effected by electroporation (see, e.g., Choi et al., 2006, J. Microbiol. Methods 64: 391-397) or by conjugation (see, e.g., Pinedo and Smets, 2005, Appl. Environ. Microbiol. 71: 51-57). The introduction of DNA into a Streptococcus cell may, for instance, be effected by natural competence (see, e.g., Perry and Kuramitsu, 1981, Infect. Immun. 32: 1295-1297), by protoplast transformation (see, e.g., Catt and Jollick, 1991, Microbios. 68: 189-2070, by electroporation (see, e.g., Buckley et al., 1999, Appl. Environ. Microbiol. 65: 3800-3804) or by conjugation (see, e.g., Clewell, 1981, Microbiol. Rev. 45: 409-436). However, any method known in the art for introducing DNA into a host cell can be used.

The host cell may also be a eukaryote, such as a mammalian, insect, plant, or fungal cell.

In a preferred aspect, the host cell is a fungal cell. “Fungi” as used herein includes the phyla Ascomycota, Basidiomycota, Chytridiomycota, and Zygomycota (as defined by Hawksworth et al., In, Ainsworth and Bisby's Dictionary of The Fungi, 8th edition, 1995, CAB International, University Press, Cambridge, UK) as well as the Oomycota (as cited in Hawksworth et al., 1995, supra, page 171) and all mitosporic fungi (Hawksworth et al., 1995, supra).

In a more preferred aspect, the fungal host cell is a yeast cell. “Yeast” as used herein includes ascosporogenous yeast (Endomycetales), basidiosporogenous yeast, and yeast belonging to the Fungi Imperfecti (Blastomycetes). Since the classification of yeast may change in the future, for the purposes of this invention, yeast shall be defined as described in Biology and Activities of Yeast (Skinner, F. A., Passmore, S. M., and Davenport, R. R., eds, Soc. App. Bacteriol. Symposium Series No. 9, 1980).

In an even more preferred aspect, the yeast host cell is a Candida, Hansenula, Kluyveromyces, Pichia, Saccharomyces, Schizosaccharomyces, or Yarrowia cell.

In a most preferred aspect, the yeast host cell is a Saccharomyces carlsbergensis, Saccharomyces cerevisiae, Saccharomyces diastaticus, Saccharomyces douglasii, Saccharomyces kluyveri, Saccharomyces norbensis, or Saccharomyces oviformis cell. In another most preferred aspect, the yeast host cell is a Kluyveromyces lactis cell. In another most preferred aspect, the yeast host cell is a Yarrowia lipolytica cell.

In another more preferred aspect, the fungal host cell is a filamentous fungal cell. “Filamentous fungi” include all filamentous forms of the subdivision Eumycota and Oomycota (as defined by Hawksworth et al., 1995, supra). The filamentous fungi are generally characterized by a mycelial wall composed of chitin, cellulose, glucan, chitosan, mannan, and other complex polysaccharides. Vegetative growth is by hyphal elongation and carbon catabolism is obligately aerobic. In contrast, vegetative growth by yeasts such as Saccharomyces cerevisiae is by budding of a unicellular thallus and carbon catabolism may be fermentative.

In an even more preferred aspect, the filamentous fungal host cell is an Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, or Trichoderma cell.

In a most preferred aspect, the filamentous fungal host cell is an Aspergillus awamori, Aspergillus fumigatus, Aspergillus foetidus, Aspergillus japonicus, Aspergillus nidulans, Aspergillus niger or Aspergillus oryzae cell. In another most preferred aspect, the filamentous fungal host cell is a Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, or Fusarium venenatum cell. In another most preferred aspect, the filamentous fungal host cell is a Bjerkandera adusta, Ceriporiopsis aneirina, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Chrysosporium keratinophilum, Chrysosporium lucknowense, Chrysosporium tropicum, Chrysosporium merdarium, Chrysosporium inops, Chrysosporium pannicola, Chrysosporium queenslandicum, Chrysosporium zonatum, Coprinus cinereus, Coriolus hirsutus, Humicola insolens, Humicola lanuginosa, Mucor miehei, Myceliophthora thermophila, Neurospora crassa, Penicillium purpurogenum, Phanerochaete chtysosporium, Phlebia radiata, Pleurotus eryngii, Thielavia terrestris, Trametes villosa, Trametes versicolor, Trichoderma harzianum, Trichoderma koningii, Trichoderma longibrachiatum, Trichoderma reesei, or Trichoderma viride cell.

Fungal cells may be transformed by a process involving protoplast formation, transformation of the protoplasts, and regeneration of the cell wall in a manner known per se. Suitable procedures for transformation of Aspergillus and Trichoderma host cells are described in EP 238 023 and Yelton et al., 1984, Proceedings of the National Academy of Sciences USA 81: 1470-1474. Suitable methods for transforming Fusarium species are described by Malardier et al., 1989, Gene 78: 147-156, and WO 96/00787. Yeast may be transformed using the procedures described by Becker and Guarente, In Abelson, J. N. and Simon, M. I., editors, Guide to Yeast Genetics and Molecular Biology, Methods in Enzymology, Volume 194, pp 182-187, Academic Press, Inc., New York; Ito et al., 1983, Journal of Bacteriology 153: 163; and Hinnen et al., 1978, Proceedings of the National Academy of Sciences USA 75: 1920.

### Methods of Production

The present invention also relates to methods of producing a polypeptide of the present invention, comprising: (a) cultivating a cell, which in its wild-type form produces the polypeptide, under conditions conducive for production of the polypeptide; and (b) recovering the polypeptide. In a preferred aspect, the cell is of the genus Pseudoalteromonas. In a more preferred aspect, the cell is Pseudoalteromonas haloplanktis. In a most preferred aspect, the cell is Pseudoalteromonas haloplanktis NCIMB 1964.

The present invention also relates to methods of producing a polypeptide of the present invention, comprising: (a) cultivating a recombinant host cell, as described herein, under conditions conducive for production of the polypeptide; and (b) recovering the polypeptide.

The present invention also relates to methods of producing a polypeptide of the present invention, comprising: (a) cultivating a recombinant host cell under conditions conducive for production of the polypeptide, wherein the host cell comprises a mutant nucleotide sequence having at least one mutation in the mature polypeptide coding sequence of SEQ ID NO: 1, wherein the mutant nucleotide sequence encodes a polypeptide that comprises or consists of the mature polypeptide of SEQ ID NO: 2, and (b) recovering the polypeptide.

In the production methods of the present invention, the cells are cultivated in a nutrient medium suitable for production of the polypeptide using methods well known in the art. For example, the cell may be cultivated by shake flask cultivation, and small-scale or large-scale fermentation (including continuous, batch, fed-batch, or solid state fermentations) in laboratory or industrial fermentors performed in a suitable medium and under conditions allowing the polypeptide to be expressed and/or isolated. The cultivation takes place in a suitable nutrient medium comprising carbon and nitrogen sources and inorganic salts, using procedures known in the art. Suitable media are available from commercial suppliers or may be prepared according to published compositions (e.g., in catalogues of the American Type Culture Collection). If the polypeptide is secreted into the nutrient medium, the polypeptide can be recovered directly from the medium. If the polypeptide is not secreted into the medium, it can be recovered from cell lysates.

The polypeptides may be detected using methods known in the art that are specific for the polypeptides. These detection methods may include use of specific antibodies, formation of an enzyme product, or disappearance of an enzyme substrate. For example, an enzyme assay may be used to determine the activity of the polypeptide as described herein.

The resulting polypeptide may be recovered using methods known in the art. For example, the polypeptide may be recovered from the nutrient medium by conventional procedures including, but not limited to, centrifugation, filtration, extraction, spray-drying, evaporation, or precipitation.

The polypeptides of the present invention may be purified by a variety of procedures known in the art including, but not limited to, chromatography (e.g., ion exchange, affinity, hydrophobic, chromatofocusing, and size exclusion), electrophoretic procedures (e.g., preparative isoelectric focusing), differential solubility (e.g., ammonium sulfate precipitation), SDS-PAGE, or extraction (see, e.g., Protein Purification, J.-C. Janson and Lars Ryden, editors, VCH Publishers, New York, 1989) to obtain substantially pure polypeptides.

### Compositions

The present invention also relates to compositions comprising a polypeptide of the present invention. Preferably, the compositions are enriched in such a polypeptide. The term “enriched” indicates that the organophosphorous hydrolase activity of the composition has been increased, e.g., with an enrichment factor of at least 1.1.

The composition may comprise a polypeptide of the present invention as the major enzymatic component, e.g., a mono-component composition. Alternatively, the composition may comprise multiple enzymatic activities, such as an aminopeptidase, amylase, carbohydrase, carboxypeptidase, catalase, cellulase, chitinase, cutinase, cyclodextrin glycosyltransferase, deoxyribonuclease, esterase, alpha-galactosidase, beta-galactosidase, glucoamylase, alpha-glucosidase, beta-glucosidase, haloperoxidase, invertase, laccase, lipase, mannosidase, oxidase, pectinolytic enzyme, peptidoglutaminase, peroxidase, phytase, polyphenoloxidase, proteolytic enzyme, ribonuclease, transglutaminase, or xylanase. The additional enzyme(s) may be produced, for example, by a microorganism belonging to the genus Aspergillus, preferably Aspergillus aculeatus, Aspergillus awamori, Aspergillus fumigatus, Aspergillus foetidus, Aspergillus japonicus, Aspergillus nidulans, Aspergillus niger, or Aspergillus oryzae; Fusarium, preferably Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sulphureum, Fusarium toruloseum, Fusarium trichothecioides, or Fusarium venenatum; Humicola, preferably Humicola insolens or Humicola lanuginosa; or Trichoderma, preferably Trichoderma harzianum, Trichoderma koningii, Trichoderma longibrachiatum, Trichoderma reesei, or Trichoderma viride.

The polypeptide compositions may be prepared in accordance with methods known in the art and may be in the form of a liquid or a dry composition. For instance, the polypeptide composition may be in the form of a granulate or a microgranulate. The polypeptide to be included in the composition may be stabilized in accordance with methods known in the art.

Examples are given below of preferred uses of the polypeptide compositions of the invention. The dosage of the polypeptide composition of the invention and other conditions under which the composition is used may be determined on the basis of methods known in the art.

### Uses

The present invention is also directed to methods for using the polypeptides having organophosphorous hydrolase activity, or compositions thereof.

In one preferred embodiment the invention also directed to the use of organophosphorous hydrolase of the invention for decontamining an area or a decive contaminated with at least one harmful or undesired organophosphorous compound. The organophosphorous hydrolase of the invention or a composition comprising the organophosphorous hydrolase of the invention is applied to the area or the device in an amount sufficient to degrade at least part of the at least one harmful or undesired organophosphorous compound.

Harmful or undesired organophosphorous compounds includes toxic organophosphorous cholinesterase-inhibiting compounds including nerve gases such as diisopropyl fluoro phosphate (DFP), O-isopropyl methylphosphonofluoridate (sarin), O-pinacolyl methyl phosphonofluoridate (soman) and O-cyclohexyl methylphosphonofluoridate.

The present invention is further described by the following examples that should not be construed as limiting the scope of the invention.

### EXAMPLES

Chemicals used as buffers and substrates were commercial products of at least reagent grade.

### Media and Solutions

### Reagents and Media

| Nutrient agar | Peptone | 5.0 | g |
|---|---|---|---|
| | Meat extract | 3.0 | g |
| | Agar | 15.0 | g |
| | Distilled water | 1000.0 | mL |
| | Adjust pH to 7.0. | | |
| | Autoclave at 121° C., 16 minutes | | |
| Nutrient broth: | Peptone | 5.0 | g |
| | Meat extract | 3.0 | g |
| | Distilled water | 1000.0 | mL |
| | Adjust pH to 7.0. | | |
| | Autoclave at 121° C., 16 minutes | | |
| TE | 10 mM Tris-HCl, pH 7.4 | | |
| | 1 mM EDTA, pH 8.0 | | |
| TEL | 50 mg/mL Lysozym in TE-buffer | | |
| Thiocyanate | 5 M guanidium thiocyanate, | | |
| | 100 mM EDTA, and | | |
| | 0.6% w/v N-laurylsarcosine, sodium salt | | |
| | Dissolve 60 g guanidium thiocyanate and 20 mL 0.5 M | | |
| | EDTA, pH 8.0 in 20 mL H₂O at 65° C. Cool down to | | |
| | room temperature (RT) and add 0.6 g N- | | |
| | laurylsarcosine. Add H₂O to 100 mL | | |
| | and filter it through a 0.2 μm sterile filter. | | |
| NH₄Ac | 7.5 M CH₃COONH₄ | | |
| TER | 1 μg/mL RNAse A in TE-buffer | | |
| CIA | Chloroform/isoamyl alcohol 24:1 | | |
| TY*2 medium | Tryptone | 40 | g |
| | Yeast Extract | 10 | g |
| | 1% Ferrochloride | 1.4 | mL |
| | 1% Mangan(II)-chloride | 0.2 | mL |
| | 1% Magnesiumsulfate | 3.0 | mL |
| | Add destilled water up to 1000 mL | | |
| | Adjust pH to 7.3 and autoclave at 121° C., 16 minutes. | | |
| Lysisbuffer | HEPES 100 mM pH 7 | 200 | μL |
| | Triton X-100 | 20 | μL |
| | DNAse 10 mg/mL | 2 | μL |
| | Lysozym 10 mg/mL | 2 | μL |

Add destilled water up to 1 mL

DNAse 10 mg/mL in 50% glycerol and 10 mM Hepes pH 7

### Example 1

### Cloning and Expression of Organophosphorus Acid Anhydrase Gene, opaA, from Pseudoalteromonas haloplanktis NCIMB 1964

### Cloning of the Gene

Genomic DNA from Pseudoalteromonas haloplanktis NCIMB 1964 was isolated according to the following procedure from an over night culture in nutrient broth at 37° C.:

1. Harvest 1.5 mL culture and resuspend in 100 μL TEL. Incubate at 37° C. for 30 min.
2. Add 500 μL thiocynate buffer and leave at room temperature for 10 min.
3. Add 250 μL NH₄Ac and leave at ice for 10 min.

### 4. Add 500 μL CIA and mix.

5. Transfer to a microcentrifuge and spin for 10 min. at full speed.
6. Transfer supernatant to a new Eppendorf tube and add 0.54 volume cold isopropanol. Mix thoroughly.
7. Spin and wash the DNA pellet with 70% EtOH.
8. Resuspend the genomic DNA in 100 μL TER.

The genomic DNA from Pseudoalteromonas haloplanktis NCIMB 1964 was used as template for PCR amplification of the opaA Pseudoalteromonas haloplanktis NCIMB 1964 gene by standard PCR methods using primer 1899 (SEQ ID NO: 3) and primer 1900 (SEQ ID NO: 4).

Primer 1899:
(SEQ ID NO: 3)
5′-GGAATTCCATATGGATAAATTAGCGGTGCTATAC-3′
(start codon in bold and restriction site Nde I
is underlined)
Primer 1900:
(SEQ ID NO: 4)
5′-CCGCTCGAGATCTAAGTGTAGATCACGCGTCAT-3′
(restriction site Xho I is underlined)

Please note that using primer 1900, the stop codon TGA is removed, making the amplified DNA useful for expression with C-terminal His tags.

The PCR product amplified with primer 1899 and 1900 was digested with restriction enzyme Nde I and Xho I using standard method, and the resulting fragment was ligated to Nde I/Xho I digested pET-30a(+) (Novagen) vector prior to transformation into E. coli DH10B. E. coli DH10B Kanamycin resistant transformants were further analyzed by DNA sequencing of the opaA gene insert in the pET-30a(+). A plasmid with correct sequence, pOPAANN10719-1, was selected.

The cloned DNA was sequences and the DNA sequence encoding the opaA, from Pseudoalteromonas haloplanktis NCIMB 1964 is shown in SEQ ID NO: 1. The deduced amino acid sequence is shown in SEQ ID NO: 2.

The amino acid sequence was 91.14% identical to the prior art polypeptide having the Entry Name UNIPROT:P77814 in the UNIPOOT database.

### Fermentation

pOPAANN10719-1 was transformed into E. coli BL21 according to the pET System Manual (July 2002). A transformant, E. coli BL21 (pOPAANN10719-1), was selected for expression.

Recombinant expression of the His taq OPAA from Pseudoalteromonas haloplanktis NCIMB 1964 was done from E. coli BL21 (pOPAANN10719-1) according to the pET System Manual.

### Lysis of Cells

Cells were harvested at 4000 rpm, 30 min. Cell pellet was resuspended in lysis buffer (a 1/10 of the culture volume) and transferred to 37° C. under shaking (250 rpm) for 15 minutes. The samples were centrifuged (4000 rpm, 10 minutes) and the supernatants were collected.

### Example 2

### Purification of OPAA

The lysed sample solution was filtrated through a Fast PES bottle top filter with a 0.22 micrometer cut-off.

The solution were added solid NaCl, NaH₂PO₄.2H₂O and imidazole giving a solution with the following approximate concentrations (50 mM NaH₂PO₄, 20 mM imidazole and 0.5 M NaCl). The pH of the resulting solution was adjusted to 7.4 and the polypeptide was purified using a chelating sepharose FF column preloaded with Cu²⁺ using an Akta purifier 900 system. The elution was performed step-wise with increasing imidazole concentrations [0, 10%, 20% and 50% of (50 mM NaH₂PO₄, 500 mM imidazole, 0.5 M NaCl)]. Fractions belonging to the peak eluted with 50% imidazole were pooled, concentrated and buffer-changed into 50 mM Na-phosphate pH 7.0 using Amicon ultra-15 centrifugal filter devices with a 30 kDa cut-off membrane.

The molecular weight, as estimated from SDS-PAGE, was approximately 50 kDa and the purity was >95%.

### Example 3

### Mass Spectrometric Analysis of OPAA from P. haloplanktis

The molecular weight of the OPAA molecule was determined by an electrospray ionization mass spectrometric (ESI-MS) experiment. The spectrum showed that the preparation is of very high purity with a major peak at 51429.57 m/z.

### Example 4

### Characterisation of the P. haloplanktis OPAA

### OPAA Metal Binding

In order to examine metal ion binding samples of OPAA from P. haloplanktis was analysed of Na, K, Ca, Mg, Al, Fe, Zn, Mn, and B using Inductively Coupled Plasma-Optical Emission Spectroscopy (ICP-OES). The results from this analysis can be seen in the table below.

TABLE 1
Results from Inductively Coupled Plasma-Optical
Emission Spectroscopy

| | P. haloplanktis OPAA |
|---|---|
| Protein concentration (μM) | 235 |
| Al (mg/kg) | 3.6 |
| B (mg/kg) | 5 |
| Ca (mg/kg) | 1.5 |
| Fe (mg/kg) | 1.4 |
| K (mg/kg) | 1 |
| Mn (mg/kg) | 17 |
| Zn (mg/kg) | 3.4 |

For the OPAA, a ratio of 1.3 Mn atoms per protein molecules can be calculated. Since no Mn was added during or after the purification it is concluded that the OPAA contains Mn (probably 2 Mn per molecule). It has previously been found that other OPAAs are Mn-dependent metalloenzymes.

### Paraoxonase Activity

The activity was determined using the commercial EnzChek® Paraoxonase Assay Kit from Invitrogen. The assay is a highly sensitive fluorometric assay (excitation/emission 360/450 nm) for the organophosphatase activity of paraoxonase, based on the hydrolysis of a fluorogenic organophosphate analog. The organophosphatase activity confers protection against toxic organophosphates, and the assay may therefore be used to indicate acitivity against organophosphate-based chemical warfare agents. A standard curve for fluorescent reference standard allows determination of the amount of product formed per minute.

Typically, fermentation broth was diluted 25-50 times prior to analysis. Ten μL diluted sample was then transferred to a 96-well microplate and added 50 μL reaction buffer. The reaction was initiated by addition of 50 μL of 2× paraoxonase substrate. The hydrolysis reaction was monitored in a fluorescence microplate reader at room temperature. Positive and negative controls with/without paraoxonase were always included in each experiment. The background fluorescence of the negative control was substrated from all other samples.

The obtained data confirm that the enzyme is active and a Paraoxonase activity using the Invitro kit of the purified OPAA derived from P. haloplanktis of 56 nmol/min/μL was found.

### Determination of pH Activity Profile Using Paraoxonase Activity

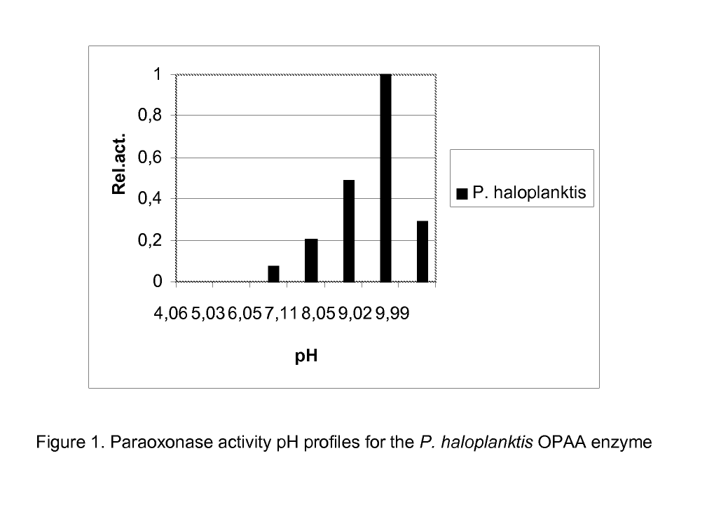

The pH dependence of the enzymatic activity of OPAA from P. haloplanktis was determined using the paraoxonase activity assay described above. However, the buffer normally used in the assay was substituted with Britton-Robinson buffers. The buffers consisted of phosphoric acid, acetic acid, and boric acid, and the pH was adjusted to the desired value by addition of sodium hydroxide prior to use. The final buffer concetration in the assay was 25 mM. Results are shown in FIG. 1. The data has been corrected for the autohydrolysis observed under alkaline conditions.

### Stabilisation of OPAAs

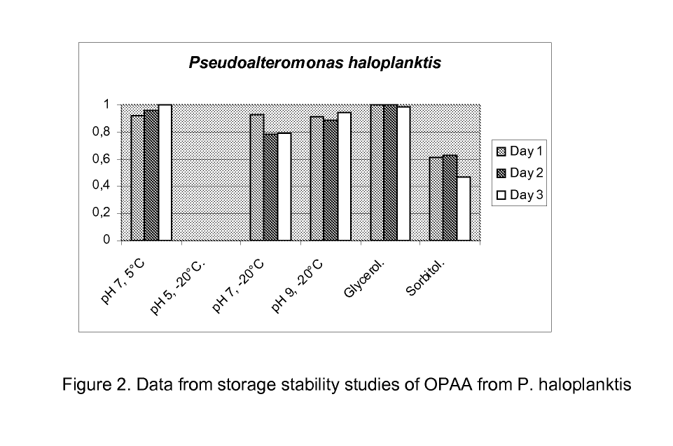

Storage of the purified protein frozen at −20° C. proved to be a problem, as heavy precipitation was observed upon thawing of the samples. It was not possible to get the precipitated protein back in solution. Solutions have therefore been studied to find methods for formulation and stabilization of the purified protein during freeze-thaw cycles. The effect of several additives was tested by monitoring the degree of precipitation and the residual activity in samples after repeated cycles of freeze-thaw. The results are presented in FIG. 2. The problem of precipitation obviously did not exist when storing the protein at 5° C. and pH 7, but the method is not useful for long term storage of the protein. The precipitation was very pronouced when the protein was stored frozen in buffer at neutral or acidic pH, while no precipitation was observed when the protein was stored frozen in a buffer under alkaline conditions (pH 9). Addition of 10% glycerol to buffer pH 7 also prevented precipitation upon freeze-thaw. Furthermore, it was observed that only this method of preservation retained full enzymatic activity during the course of the experiment. The sugar alcohol, sorbitol, was also tested as an alternative to glycerol, but this additive did not result in the same degree of stabilisation as observed for glycerol.

### Activity Against G-Agents

### Storage and Treatment of Enzyme Samples

Enzyme stock solutions were stored at −20° C. For analytical purposes the stock solution was diluted to final concentrations of 0.5 and 1 mg/mL in 20 mM Tris Buffer, pH 8.0. These diluted solutions were stored at +10° C. and used within a week.

### Assay Conditions and Enzyme Stability

Trials were conducted using a pH-Stat apparatus (titration with NaOH). The reaction mixture contained 10 mM NaCl. Substrate was added dissolved in acetonitrile (2% solution) so that the organic solvent content was 10%. In a pretitration the pH was set to 8.0 and the reaction started by the addition of 2 μL of enzyme solution (0.5 mg/mL).

In contrast to DFPase it turned out that the enzyme of the invention was unstable in the presence of 10% acetonitrile and a clear loss of activity was seen just after the start of the reaction. To overcome these difficulties substrate was dissolved in isopropanol instead of acetonitrile. NaOH consumption was constant over a period of at least 15 min and therefore the enzymes were stable under these reaction conditions.

Activity against paraoxon and phenylacetate were measured using a photospectrometer detecting the reaction products p-nitrophenol and phenol (at wavelength 405 and 280 nm). The reaction mixture contained 50 mM Tris Buffer, pH 8.0.

### Qualitative Activity Test

The OPAA was tested qualitatively with the following G-agents: Soman (GD), Tabun (GA), Cyclosarin (GF) and Sarin (GB) (Test kindly performed by Dr Marc-Michael Blum from Institut für Pharmakologie and Toxikologie der Bundeswehr). In addition to this also paraoxon and phenylacetate were tested. It has to be noted that the quality of Sarin was only good enough for qualitative results but not for quantification. The OPAA showed activity against all four G-agents and paraoxon but no activity was found for phenylacetate as a substrate.

### Activity Test and Kinetics

The specific activities were determined for a number of substrates and the results are summarized in table 2. Enzyme activities were determined by pH-Stat titration with 0.01 M NaOH. Agent was used from a 1% stock solution in isopropanol so that the final reaction mixture contained 10% isopropanol and 90% water and the final agent concentration was 0.1%. All the activities meassured were the results from at least three independent meassurments.

TABLE 2
Results on evaluation of enzymes on live nerve agents. The assays were performed
by Dr Marc-Michael Blum from Institut für Pharmakologie und Toxikologie der Bundeswehr.

| | 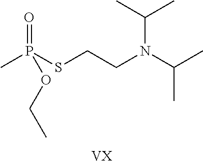 | 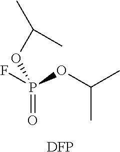 | 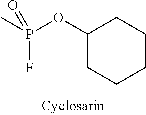 | 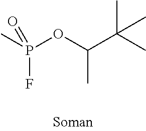 |
|---|---|---|---|---|
| Pseudoalteromonas | 0% | 100% | 38% | 49% |
| haloplanktis OPAA | | | | |
| Loligo vulgaris | 0% | 66% | 50% | 34% |
| DFPase | | | | |

The results shows that the enzyme of the invention has high activity on several G-agents and has significantly higher activity on DFP and Soman compared to the well characterized DFPase from Loligo vulgaris.

In addition, kinetic values were determined for the Pseudoalteromonas haloplanktis OPAA with Soman:

### Kₘ (mM): 0.89

kcat (s⁻¹): 1360

1. An isolated polypeptide having organophosphorous hydrolase activity, selected from the group consisting of:
(a) a polypeptide comprising an amino acid sequence having preferably at least 92%, more preferably at least 93%, more preferably at least 94%, more preferably at least 95%, more preferably at least 96%, more preferably at least 97%, even more preferably at least 98%, and even most preferably at least 99% identity to the mature polypeptide of SEQ ID NO: 2;
(b) a polypeptide encoded by a polynucleotide that hybridizes under high stringency conditions with (i) the mature polypeptide coding sequence of SEQ ID NO: 1, (ii) the genomic DNA comprising the mature polypeptide coding sequence of SEQ ID NO: 1, or (iii) a full-length complementary strand of (i) or (ii);
(c) a polypeptide encoded by a polynucleotide comprising a nucleotide sequence having preferably at least 90%, more preferably at least 91%, more preferably at least 92%, more preferably at least 93%, more preferably at least 94%, more preferably at least 95%, more preferably at least 96%, even more preferably at least 97%, most preferably at least 98%, and even most preferably at least 99% identity to the mature polypeptide coding sequence of SEQ ID NO: 1; and
(d) a variant comprising a substitution, deletion, and/or insertion of one or several amino acids of the mature polypeptide of SEQ ID NO: 2.

2. The polypeptide of claim 1, comprising or consisting of the amino acid sequence of SEQ ID NO: 2; or a fragment thereof having organophosphorous hydrolase activity.

3. The polypeptide of claim 2, comprising or consisting of the mature polypeptide of SEQ ID NO: 2.

4. The polypeptide of claim 1, which is encoded by a polynucleotide comprising or consisting of the nucleotide sequence of SEQ ID NO: 1; or a subsequence thereof encoding a fragment having organophosphorous hydrolase activity.

5. The polypeptide of claim 4, which is encoded by a polynucleotide comprising or consisting of the mature polypeptide coding sequence of SEQ ID NO: 1.

6. An isolated polynucleotide comprising a nucleotide sequence that encodes the polypeptide of claim 1.

7. The nucleic acid construct comprising the polynucleotide of claim 6 operably linked to one or more control sequences that direct the production of the polypeptide in an expression host.

8. The nucleic acid construct of claim 7, comprising a gene encoding a protein operably linked to one or both of a first nucleotide sequence encoding a signal peptide comprising or consisting of amino acids 1 to 440 of SEQ ID NO: 2, and a second nucleotide sequence encoding a propeptide comprising or consisting of amino acids 1 to 440 of SEQ ID NO: 2, wherein the gene is foreign to the first and second nucleotide sequences.

9. A recombinant expression vector comprising the nucleic acid construct of claim 7.

10. A recombinant host cell comprising the nucleic acid construct of claim 7.

11. A method of producing the polypeptide of claim 1, comprising: (a) cultivating a cell, which in its wild-type form produces the polypeptide, under conditions conducive for production of the polypeptide; and (b) recovering the polypeptide.

12. A method of producing the polypeptide of claim 1, comprising: (a) cultivating a host cell comprising a nucleic acid construct comprising a nucleotide sequence encoding the polypeptide under conditions conducive for production of the polypeptide; and (b) recovering the polypeptide.

13. A method of producing a protein, comprising: (a) cultivating the recombinant host cell of claim 10 under conditions conducive for production of the protein; and (b) recovering the protein.

14. A composition comprising a polypeptide according to claim 1.

15. A use of a polypeptide of claim 1 for decontamining an area or a device contaminated with at least one harmful or undesired organophosphorous compound, preferably, wherein the at least one harmful or undesired organophosphorous compound is selected among G-agents and pesticides, such as Soman, Tabun, Sarin and Cyclosarin.


SEQUENCE LISTING (4 sequences)

SEQ ID NO: 1 - DNA, 1323 bp, Pseudoalteromonas haloplanktis
  CDS (1)..(1323)
atggataaattagcggtgctatacgccgaacatattgcaaccttgcaacaacgcactcgtacaattacagagcgtgaaggtttagaagggttagtaattcactcaggtcaggctaaacgtcagtttttagatgacatgtattatccgtttaaggtgaatccgcaatttaaagcttggttaccagtgatcgacaacccgcattgctggattgtggttgatggggcatctaagccgaaactgattttttatcgtcctgtcgacttttggcataaagtgcctgacgaaccacgtgatttttgggctgaatattttgatattgaattattagtacagccagatcaggtagagaaactgcttccttatgataaagctaaatatgcttatatcggtgaatatctagaagtagcgcaagcgctgggcttcagcataatgaacccagagccagtgatgaactacttgcattttcatcgtgcttataaaacacagtacgaacttgagtgcttacgtcaagcaaaccgtatcgcggtagatggccacaaagctgcacgtgatacgttttttgctggtggttcagagtttgatattcagcaagcttacctcatggcaacccgccagagtgaaaacgaaatgccttacggtaatatcgttgcattaaatgagaactgtgcaattctacactacacgcattttgaacctaaagcgccacaaacgcatcactctttccttattgatgctggtgcgaactttaatggttatgctgctgatatcacacgtacttatgactttaaaaagtcaggtgaatttagcgacttaattaaagtaatgactgagcatcaaatcgctttaggtaaagcattaaagccgggcttgttatacggtgaattgcatttagattgtcatcagcgtgtagcgcaagtgctaagcgactttaatattgtgaagttaccagccgctgaaattgttgagcgtggtattacgtcgacgttcttcccgcatggtcttggtcatcaccttggtttacaagttcacgatatgggtggttttatggctgatgatacaggtgcgcaccaagcaccacctgaaggtcatccgttcctacgttgcactcgcttaatcgaaaagaaccaagtatttactattgaacctggtttatactttatcgactcgttattaggtgaccttgcgcaaactgacaacaaacagtttattaactgggaaaaagttgaagcgcttaagccatttggcggtatccgtattgaagataatattatcgttcatgaagatagcctagaaaacatgacgcgtgatctacacttagattaa

SEQ ID NO: 2 - protein, 440 aa, Pseudoalteromonas haloplanktis
MDKLAVLYAEHIATLQQRTRTITEREGLEGLVIHSGQAKRQFLDDMYYPFKVNPQFKAWLPVIDNPHCWIVVDGASKPKLIFYRPVDFWHKVPDEPRDFWAEYFDIELLVQPDQVEKLLPYDKAKYAYIGEYLEVAQALGFSIMNPEPVMNYLHFHRAYKTQYELECLRQANRIAVDGHKAARDTFFAGGSEFDIQQAYLMATRQSENEMPYGNIVALNENCAILHYTHFEPKAPQTHHSFLIDAGANFNGYAADITRTYDFKKSGEFSDLIKVMTEHQIALGKALKPGLLYGELHLDCHQRVAQVLSDFNIVKLPAAEIVERGITSTFFPHGLGHHLGLQVHDMGGFMADDTGAHQAPPEGHPFLRCTRLIEKNQVFTIEPGLYFIDSLLGDLAQTDNKQFINWEKVEALKPFGGIRIEDNIIVHEDSLENMTRDLHLD

SEQ ID NO: 3 - DNA, 34 bp, Artificial Sequence
  Primer 1899
ggaattccatatggataaattagcggtgctatac

SEQ ID NO: 4 - DNA, 33 bp, Artificial Sequence
  Primer 1900
ccgctcgagatctaagtgtagatcacgcgtcat